SEQUENCE LISTING (30 sequences)

SEQ ID NO: 1 - DNA, 549 bp, homo sapiens
atggccgaggagaacgtggacttccgcatccacgtggagaaccagacgcgggctcgggacgatgtgagccgtaagcagctgcggctgtaccagctctacagccggaccagtgggaaacacatccaggtcctgggccgcaggatcagtgcccgcggcgaggatggggacaagtatgcccagctcctagtggagacagacaccttcggtagtcaagtccggatcaagggcaaggagacggaattctacctgtgcatgaaccgcaaaggcaagctcgtggggaagcccgatggcaccagcaaggagtgtgtgttcatcgagaaggttctggagaacaactacacggccctgatgtcggctaagtactccggctggtacgtgggcttcaccaagaaggggcggccgcggaagggccccaagacccgggagaaccagcaggacgtgcatttcatgaagcgctaccccaaggggcagccggagcttcagaagcccttcaagtacacgacggtgaccaagaggtcccgtcggatccggcccacacaccctgcctga

SEQ ID NO: 2 - protein, 182 aa, homo sapiens
MAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 3 - DNA, 549 bp, mus musculus
atggccgaggagaatgtggacttccgcatccacgtggagaaccagacgcgggctcgagatgatgtgagtcggaagcagctgcgcttgtaccagctctatagcaggaccagtgggaagcacattcaagtcctgggccgtaggatcagtgcccgtggcgaggacggggacaagtatgcccagctcctagtggagacagataccttcgggagtcaagtccggatcaagggcaaggagacagaattctacctgtgtatgaaccgaaaaggcaagctcgtggggaagcctgatggtactagcaaggagtgcgtgttcattgagaaggttctggaaaacaactacacggccctgatgtctgccaagtactctggttggtatgtgggcttcaccaagaaggggcggcctcgcaagggtcccaagacccgcgagaaccagcaagatgtacacttcatgaagcgttaccccaagggacaggccgagctgcagaagcccttcaaatacaccacagtcaccaagcgatcccggcggatccgccccactcaccccggctag

SEQ ID NO: 4 - DNA, 537 bp, Artificial
  truncation mutant DNA of mouse FGF18(d4)
atggtggacttccgcatccacgtggagaaccagacgcgggctcgagatgatgtgagtcggaagcagctgcgcttgtaccagctctatagcaggaccagtgggaagcacattcaagtcctgggccgtaggatcagtgcccgtggcgaggacggggacaagtatgcccagctcctagtggagacagataccttcgggagtcaagtccggatcaagggcaaggagacagaattctacctgtgtatgaaccgaaaaggcaagctcgtggggaagcctgatggtactagcaaggagtgcgtgttcattgagaaggttctggaaaacaactacacggccctgatgtctgccaagtactctggttggtatgtgggcttcaccaagaaggggcggcctcgcaagggtcccaagacccgcgagaaccagcaagatgtacacttcatgaagcgttaccccaagggacaggccgagctgcagaagcccttcaaatacaccacagtcaccaagcgatcccggcggatccgccccactcaccccggctag

SEQ ID NO: 5 - DNA, 513 bp, Artificial
  truncation mutant DNA of mouse FGF18(d12)
atgaaccagacgcgggctcgagatgatgtgagtcggaagcagctgcgcttgtaccagctctatagcaggaccagtgggaagcacattcaagtcctgggccgtaggatcagtgcccgtggcgaggacggggacaagtatgcccagctcctagtggagacagataccttcgggagtcaagtccggatcaagggcaaggagacagaattctacctgtgtatgaaccgaaaaggcaagctcgtggggaagcctgatggtactagcaaggagtgcgtgttcattgagaaggttctggaaaacaactacacggccctgatgtctgccaagtactctggttggtatgtgggcttcaccaagaaggggcggcctcgcaagggtcccaagacccgcgagaaccagcaagatgtacacttcatgaagcgttaccccaagggacaggccgagctgcagaagcccttcaaatacaccacagtcaccaagcgatcccggcggatccgccccactcaccccggctag

SEQ ID NO: 6 - DNA, 501 bp, Artificial
  truncation mutant DNA of mouse FGF18(d16)
atggctcgagatgatgtgagtcggaagcagctgcgcttgtaccagctctatagcaggaccagtgggaagcacattcaagtcctgggccgtaggatcagtgcccgtggcgaggacggggacaagtatgcccagctcctagtggagacagataccttcgggagtcaagtccggatcaagggcaaggagacagaattctacctgtgtatgaaccgaaaaggcaagctcgtggggaagcctgatggtactagcaaggagtgcgtgttcattgagaaggttctggaaaacaactacacggccctgatgtctgccaagtactctggttggtatgtgggcttcaccaagaaggggcggcctcgcaagggtcccaagacccgcgagaaccagcaagatgtacacttcatgaagcgttaccccaagggacaggccgagctgcagaagcccttcaaatacaccacagtcaccaagcgatcccggcggatccgccccactcaccccggctag

SEQ ID NO: 7 - DNA, 495 bp, Artificial
  truncation mutant DNA of mouse FGF18(d18)
atggatgatgtgagtcggaagcagctgcgcttgtaccagctctatagcaggaccagtgggaagcacattcaagtcctgggccgtaggatcagtgcccgtggcgaggacggggacaagtatgcccagctcctagtggagacagataccttcgggagtcaagtccggatcaagggcaaggagacagaattctacctgtgtatgaaccgaaaaggcaagctcgtggggaagcctgatggtactagcaaggagtgcgtgttcattgagaaggttctggaaaacaactacacggccctgatgtctgccaagtactctggttggtatgtgggcttcaccaagaaggggcggcctcgcaagggtcccaagacccgcgagaaccagcaagatgtacacttcatgaagcgttaccccaagggacaggccgagctgcagaagcccttcaaatacaccacagtcaccaagcgatcccggcggatccgccccactcaccccggctag

SEQ ID NO: 8 - DNA, 483 bp, Artificial
  truncation mutant DNA of mouse FGF18(d22)
atgcggaagcagctgcgcttgtaccagctctatagcaggaccagtgggaagcacattcaagtcctgggccgtaggatcagtgcccgtggcgaggacggggacaagtatgcccagctcctagtggagacagataccttcgggagtcaagtccggatcaagggcaaggagacagaattctacctgtgtatgaaccgaaaaggcaagctcgtggggaagcctgatggtactagcaaggagtgcgtgttcattgagaaggttctggaaaacaactacacggccctgatgtctgccaagtactctggttggtatgtgggcttcaccaagaaggggcggcctcgcaagggtcccaagacccgcgagaaccagcaagatgtacacttcatgaagcgttaccccaagggacaggccgagctgcagaagcccttcaaatacaccacagtcaccaagcgatcccggcggatccgccccactcaccccggctag

SEQ ID NO: 9 - DNA, 438 bp, Artificial
  truncation mutant DNA of mouse FGF18(d37)
atgaagcacattcaagtcctgggccgtaggatcagtgcccgtggcgaggacggggacaagtatgcccagctcctagtggagacagataccttcgggagtcaagtccggatcaagggcaaggagacagaattctacctgtgtatgaaccgaaaaggcaagctcgtggggaagcctgatggtactagcaaggagtgcgtgttcattgagaaggttctggaaaacaactacacggccctgatgtctgccaagtactctggttggtatgtgggcttcaccaagaaggggcggcctcgcaagggtcccaagacccgcgagaaccagcaagatgtacacttcatgaagcgttaccccaagggacaggccgagctgcagaagcccttcaaatacaccacagtcaccaagcgatcccggcggatccgccccactcaccccggctag

SEQ ID NO: 10 - DNA, 405 bp, Artificial
  truncation mutant DNA of mouse FGF18(d48)
atggcccgtggcgaggacggggacaagtatgcccagctcctagtggagacagataccttcgggagtcaagtccggatcaagggcaaggagacagaattctacctgtgtatgaaccgaaaaggcaagctcgtggggaagcctgatggtactagcaaggagtgcgtgttcattgagaaggttctggaaaacaactacacggccctgatgtctgccaagtactctggttggtatgtgggcttcaccaagaaggggcggcctcgcaagggtcccaagacccgcgagaaccagcaagatgtacacttcatgaagcgttaccccaagggacaggccgagctgcagaagcccttcaaatacaccacagtcaccaagcgatcccggcggatccgccccactcaccccggctag

SEQ ID NO: 11 - DNA, 348 bp, Artificial
  truncation mutant DNA of mouse FGF18(d67)
atggggagtcaagtccggatcaagggcaaggagacagaattctacctgtgtatgaaccgaaaaggcaagctcgtggggaagcctgatggtactagcaaggagtgcgtgttcattgagaaggttctggaaaacaactacacggccctgatgtctgccaagtactctggttggtatgtgggcttcaccaagaaggggcggcctcgcaagggtcccaagacccgcgagaaccagcaagatgtacacttcatgaagcgttaccccaagggacaggccgagctgcagaagcccttcaaatacaccacagtcaccaagcgatcccggcggatccgccccactcaccccggctag

SEQ ID NO: 12 - DNA, 318 bp, Artificial
  truncation mutant DNA of mouse FGF18(d77)
atgacagaattctacctgtgtatgaaccgaaaaggcaagctcgtggggaagcctgatggtactagcaaggagtgcgtgttcattgagaaggttctggaaaacaactacacggccctgatgtctgccaagtactctggttggtatgtgggcttcaccaagaaggggcggcctcgcaagggtcccaagacccgcgagaaccagcaagatgtacacttcatgaagcgttaccccaagggacaggccgagctgcagaagcccttcaaatacaccacagtcaccaagcgatcccggcggatccgccccactcaccccggctag

SEQ ID NO: 13 - DNA, 264 bp, Artificial
  truncation mutant DNA of mouse FGF18(d95)
atgggtactagcaaggagtgcgtgttcattgagaaggttctggaaaacaactacacggccctgatgtctgccaagtactctggttggtatgtgggcttcaccaagaaggggcggcctcgcaagggtcccaagacccgcgagaaccagcaagatgtacacttcatgaagcgttaccccaagggacaggccgagctgcagaagcccttcaaatacaccacagtcaccaagcgatcccggcggatccgccccactcaccccggctag

SEQ ID NO: 14 - protein, 182 aa, mus musculus
MAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQAELQKPFKYTTVTKRSRRIRPTHPG

SEQ ID NO: 15 - protein, 178 aa, Artificial
  truncation mutant polypeptide of mouse FGF18(d4)
MVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQAELQKPFKYTTVTKRSRRIRPTHPG

SEQ ID NO: 16 - protein, 170 aa, Artificial
  truncation mutant polypeptide of mouse FGF18 (d12)
MNQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQAELQKPFKYTTVTKRSRRIRPTHPG

SEQ ID NO: 17 - protein, 166 aa, Artificial
  truncation mutant polypeptide of mouse FGF18(d16)
MARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQAELQKPFKYTTVTKRSRRIRPTHPG

SEQ ID NO: 18 - protein, 164 aa, Artificial
  truncation mutant polypeptide of mouse FGF18(d18)
MDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQAELQKPFKYTTVTKRSRRIRPTHPG

SEQ ID NO: 19 - protein, 160 aa, Artificial
  truncation mutant polypeptide of mouse FGF18(d22)
MRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQAELQKPFKYTTVTKRSRRIRPTHPG

SEQ ID NO: 20 - protein, 145 aa, Artificial
  truncation mutant polypeptide of mouse FGF18(d37)
MKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQAELQKPFKYTTVTKRSRRIRPTHPG

SEQ ID NO: 21 - protein, 134 aa, Artificial
  truncation mutant polypeptide of moouse FGF18(d48)
MARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQAELQKPFKYTTVTKRSRRIRPTHPG

SEQ ID NO: 22 - protein, 115 aa, Artificial
  truncation mutant polypeptide of moue FGF18(d67)
MGSQVRIKGKETEFYLCMNRKGKLVGKPDGTSKECVFIEKVLENNYTALMSAKYSGWYVGFTKKGRPRKGPKTRENQQDVHFMKRYPKGQAELQKPFKYTTVTKRSRRIRPTHPG

SEQ ID NO: 23 - protein, 105 aa, Artificial
  truncation mutant polypeptide of mouse FGF18(d77)
MTEFYLCMNRKGKLVGKPDGTSKECVFIEKVLENNYTALMSAKYSGWYVGFTKKGRPRKGPKTRENQQDVHFMKRYPKGQAELQKPFKYTTVTKRSRRIRPTHPG

SEQ ID NO: 24 - protein, 87 aa, Artificial
  truncation mutant polypeptide of mouse FGF18(d95)
MGTSKECVFIEKVLENNYTALMSAKYSGWYVGFTKKGRPRKGPKTRENQQDVHFMKRYPKGQAELQKPFKYTTVTKRSRRIRPTHPG

SEQ ID NO: 25 - DNA, 525 bp, Artificial
  truncation mutant DNA of mouse FGF18(dc8)
atggccgaggagaatgtggacttccgcatccacgtggagaaccagacgcgggctcgagatgatgtgagtcggaagcagctgcgcttgtaccagctctatagcaggaccagtgggaagcacattcaagtcctgggccgtaggatcagtgcccgtggcgaggacggggacaagtatgcccagctcctagtggagacagataccttcgggagtcaagtccggatcaagggcaaggagacagaattctacctgtgtatgaaccgaaaaggcaagctcgtggggaagcctgatggtactagcaaggagtgcgtgttcattgagaaggttctggaaaacaactacacggccctgatgtctgccaagtactctggttggtatgtgggcttcaccaagaaggggcggcctcgcaagggtcccaagacccgcgagaaccagcaagatgtacacttcatgaagcgttaccccaagggacaggccgagctgcagaagcccttcaaatacaccacagtcaccaagcgatcccggtag

SEQ ID NO: 26 - DNA, 501 bp, Artificial
  truncation mutant DNA of mouse FGF18(dc15)
atggccgaggagaatgtggacttccgcatccacgtggagaaccagacgcgggctcgagatgatgtgagtcggaagcagctgcgcttgtaccagctctatagcaggaccagtgggaagcacattcaagtcctgggccgtaggatcagtgcccgtggcgaggacggggacaagtatgcccagctcctagtggagacagataccttcgggagtcaagtccggatcaagggcaaggagacagaattctacctgtgtatgaaccgaaaaggcaagctcgtggggaagcctgatggtactagcaaggagtgcgtgttcattgagaaggttctggaaaacaactacacggccctgatgtctgccaagtactctggttggtatgtgggcttcaccaagaaggggcggcctcgcaagggtcccaagacccgcgagaaccagcaagatgtacacttcatgaagcgttaccccaagggacaggccgagctgcagaagcccttcaaatactag

SEQ ID NO: 27 - DNA, 474 bp, Artificial
  truncation mutant DNA of mouse FGF18(dc25)
atggccgaggagaatgtggacttccgcatccacgtggagaaccagacgcgggctcgagatgatgtgagtcggaagcagctgcgcttgtaccagctctatagcaggaccagtgggaagcacattcaagtcctgggccgtaggatcagtgcccgtggcgaggacggggacaagtatgcccagctcctagtggagacagataccttcgggagtcaagtccggatcaagggcaaggagacagaattctacctgtgtatgaaccgaaaaggcaagctcgtggggaagcctgatggtactagcaaggagtgcgtgttcattgagaaggttctggaaaacaactacacggccctgatgtctgccaagtactctggttggtatgtgggcttcaccaagaaggggcggcctcgcaagggtcccaagacccgcgagaaccagcaagatgtacacttcatgaagcgttaccccaagggacagtag

SEQ ID NO: 28 - protein, 174 aa, Artificial
  truncation mutant polypeptide of mouse FGF18(dc8)
MAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQAELQKPFKYTTVTKRSR

SEQ ID NO: 29 - protein, 167 aa, Artificial
  truncation mutant polypeptide of mouse FGF18(dc15)
MAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQAELQKPFKYT

SEQ ID NO: 30 - protein, 157 aa, Artificial
  truncation mutant polypeptide of mouse FGF18(dc25)
MAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQ

(19) United States
(12) Patent Application Publication
(10) Pub. No.: US 2012/0251455 A1
(43) Pub. Date: Oct. 4, 2012

(54) Hair Growth-Inhibiting Agent

(75) Inventors: Toru IMAMURA, Ibaraki (JP); Miho KIMURA, Ibaraki (JP); Mitsuko KAWANO, Ibaraki (JP); Nozomi TSUJINO, Ibaraki (JP); Akiko KURAMOCHI, Ibaraki (JP); Yuko ODA, Ibaraki (JP); Kaori MOTOMURA, Ibaraki (JP); Masashi SUZUKI, Ibaraki (JP); Masahiro ASADA, Ibaraki (JP); Azusa KAMEYAMA, Ibaraki (JP); Sumie TOGAYACHI, Ibaraki (JP); Syuichi OKA, Ibaraki (JP)

(73) Assignee: NATIONAL INSTITUTE OF ADVANCED INDUSTRIAL SCIENCE AND TECHNOLOGY, Tokyo (JP)

(21) Appl. No.: 13/360,419

(22) Filed: Jan. 27, 2012

Related U.S. Application Data
(62) Division of application No. 12/527,693, filed on Aug. 18, 2009, now abandoned.

(30) Foreign Application Priority Data
Feb. 19, 2007 (JP) 2007-037844

(51) Int. Cl. C12Q 1/68; A61K 49/00; G01N 33/566; G01N 21/25

(52) U.S. Cl. 424/9.2; 435/6.1; 435/7.92; 435/6.12; 435/6.11

(57) ABSTRACT

A hair removing agent or a hair growth inhibiting agent is provided by determining an endogenous factor having a hair growth inhibitory activity, screening for substances having an activity similar to that of the endogenous factor or substances enhancing the activity or expression of the endogenous factor, and utilizing the physiological activities of such substances. It has been found that FGF18 inhibits hair growth. A method of screening for FGF18-like active substances, substances that promote the activity of FGF18 or substances that promote the expression of FGF18 to thereby obtain candidates for the hair growth inhibiting agent or hair removing agent is disclosed. Also disclosed is a hair growth inhibiting agent or a hair removing agent comprising, as an active ingredient, FGF18 and/or an FGF18 partial peptide, or an FGF18-like active substance and/or a substance that promotes the activity or expression of FGF18 (e.g., Digenea simplex extract).

### TECHNICAL FIELD

The present invention relates to a hair growth-inhibiting agent or a hair removing agent both comprising a substance which inhibits the growth of hair follicles (synonymous with hair roots), and a method of screening for such substances.

### BACKGROUND ART

It is known that a variety of polypeptide growth factors, including various members of the fibroblast growth factor (hereinafter, referred to as “FGF”) family, are expressed in skin tissue. In both mouse and human, FGFs are encoded by 22 distinct genes (Non-Patent Document 1). Among them, FGF1, FGF2, FGF5, FGF7, FGF10, FGF13 and FGF22 are reported to be expressed in dermal cells and hair follicular cells to regulate hair growth and skin regeneration (see Non-Patent Documents 2-17 and Patent Document 1).

Non-Patent Document 2-17 and other documents suggest that FGF plays an important role in the growth and differentiation of cutaneous cells. However, it is still unknown as to how the FGF group is involved in the effect of promoting or inhibiting the growth of hair follicles and the resultant hair growth promoting effect or hair growth inhibiting effect.

Under the above-described circumstances, the present inventors found that single administration of FGF18 to mouse skin with hair follicles in telogen phase (resting phase) induces hair regrowth, and reported that FGF18 is a substance which induces onset of anagen phase (growth phase) in follicles to result in promotion of hair growth (Non-Patent Document 19 and Patent Document 2).
Non-Patent Document 1: Ornitz D M, Itoh N: Fibroblast growth factors. Genome Biol 2: RE VIE WS3005, 2001
Non-Patent Document 2: du Cros D L: Fibroblast growth factor and epidermal growth factor in hair development. J Invest Dermatol 101: 106S-113S. 1993
Non-Patent Document 3: du Cros D L, Isaacs K, Moore G P: Distribution of acidic and basicfibr oblast growth factorsin ovine skin during follicle morphogenesis. J Cell Sci 105: 667-674, 1993
Non-Patent Document 4: Herbert J M, Rosenquist T, Gotz J, Martin G R: FGF5 as a regulator of the hair growth cycle: Evidence from targeted and spontaneous mutations. Cell 78: 1017-1025, 1994
Non-Patent Document 5: Danilenko D M, Ring B D, Yanagihara D, Benson W, Wiemann B, Starnes C O, Pierce G F: Keratinocyte growth factor is an important endogenous mediator of hair follicle growth, development, and differentiation. American J Pathol 147: 145-154, 1995
Non-Patent Document 6: Marchese C, Chedid M, Dirsch O R, et al: Modulation of keratinocyte growth factor and its receptor in reepithelializing human skin. J Exp Med 182: 1369-1376, 1995

Non-Patent Document 7: Guo L, Degenstein L, Fuchs E: keratinocyte growth factor is required for hair development but not for wound healing. Genes Dev 10: 165-175, 1996
Non-Patent Document 8: Rosenquist T A, Martin G R: Fibroblast growth factor signalling in the hair growth cycle: Expression of the fibroblast growth factor receptor and ligand genes in the murine hair follicle. Developmental Dynamics 205:379-386, 1996
Non-Patent Document 9: Petho-Schramm A, Muller H J, Paus R: FGF5 and the murine hair cycle, Arch Dermatol Res 288: 264-266, 1996
Non-Patent Document 10: Mitsui 5, Ohuchi A, Hotta M, Tsuboi R, Ogawa H: Genes for a range of growth factors and cyclin-dependent kinase inhibitors are expressed by isolated human hair follicles. Br J Dermatol 137:693-698, 1997
Non-Patent Document 11: Ortega S, Ittmann M, Tsang S H, Ehrlich M, Basilico C: Neuronal defects and delayed wound healing in mice lacking fibroblast growth factor-2. Proc Natl Acad Sci USA 95: 5672-5677, 1998
Non-Patent Document 12: Suzuki 5, Kato T, Takimoto H, et al: Localization of rat FGF-5 protein in skin macrophage-like cells and FGF-SS protein in hair follicle: Possible involvement of two Fgf-5 gene products in hair growth cycleregulation, J Invest Dermatol 111: 963-972, 1998
Non-Patent Document 13: Suzuki S, Ota Y, Ozawa K, Imamura T: Dual-mode regulation of hair growth cycle by two Fgf-5 gene products. J Invest Dermatol 114: 456-463, 2000
Non-Patent Document 14: Nakatake Y, Hoshikawa M, Asaki T, Kassai Y, Itoh N: Identification of a novel fibroblast growth factor, FGF-22, preferentially expressed in the inner root sheath of the hair follicle. Biochem Biophys Acta 1517: 460-463, 2001
Non-Patent Document 15: Steen K S, Paus R Controls of hair follicle cycling. Physiol Rev 81: 449-494, 2001
Non-Patent Document 16: Beyer T A., Werner 5, Dickson C, Grose R: Fibroblast growth factor 22 and its potential role during skin development and repair. Exp Cell Res 287: 228-236 2003
Non-Patent Document 17: kawano M, Suzuki S, Suzuki M, Old 3, Imamura T: Bulge- and basallayer-specific expression of fibroblast growth factor 13(FHF-2) in mouse skin. Invest Dermatol 122: 1084-1090, 2004
Non-Patent Document 18: Suzuki S, Ota Y, Ozawa K, Imamura T: Dual-mode regulation of hair growth cycle by two Fgf-5 gene products. J Invest Dermatol 114: 456-463, 2000
Non-Patent Document 19: Kawano M, Komi-Kuramochi A, Asada M, Suzuki M, Oki I, Jiang J, Imamura T: Comprehensive analysis of FGF and FGFR expression in skin: FGF18 is highly expressed in hair follicles and capable of inducing anagen from telogenstage hair follicles. J Invest Dermatol, 2005, 124(5): 877-885
Non-Patent Document 20: ZhangX, Ibrahimi O A, Olsen S K, Umemori H, Mohammadi M, Ornitz D M. Receptor Specificity of the Fibroblast Growth Factor Family: THE COMPLETE MAMMALIAN FGFFAMILY. J Biol Chem 2006, 281(23):15694-15700
Patent Document 1: Japanese Unexamined Patent Publication No. Nei 4-224522
Patent Document 2: Japanese Unexamined Patent Publication No. 2006-83082

### DISCLOSURE OF THE INVENTION

### Problem for Solution by the Invention

Many people are having trouble with unwanted hair. To cope with this problem, removal of the hair shaft is often carried out using chemical substances or by means of this physical methods. However, the effect is soon lost as a result of natural follicle growth. It is an object of the present invention to elucidate the mode of action of endogenous factors that regulate hair growth, to determine an endogenous factor that inhibits hair growth, and to provide a hair growth-inhibiting agent or a hair removing agent using the endogenous factor. Further, it is another object of the present invention to screen for substances that promote the activity or expression of the endogenous factor and substances that have an effect similar to the effect of the endogenous factor, and to provide those substances which have a hair growth-inhibiting effect similar to the effect of the endogenous factor.

It is still another object of the present invention to provide a screening method for the above-described purpose.

### Means to Solve the Problem

As described above, it was already confirmed that single administration of FGF18 to skin with hair follicles in telogen phase promotes hair growth after several weeks of reaction period. Therefore, FGF18 was believed to be a substance that has a hair growth promoting effect.

However, the present inventors have obtained an unexpected, surprising finding when FGF18 was administered to mouse dorsal skin subcutaneously once a day for 8 days after compulsive induction of anagen phase in follicles of the dorsal skin in telogen phase by depilating, so that FGF18 was allowed to persist in hair follicles continuously for observing the state of growth of hair.

Briefly, hair growth progressed well and follicle enlargement occurred at day 9 in the control group which received phosphate-buffered physiological saline under similar conditions whereas hair growth was markedly inhibited the FGF18 administered group.

Further, the present inventors performed a detailed functional analysis of FGF18. As a result, it was found that the expression level of a growth factor VEGF (which is considered important for hair follicle growth) decreased when dermal papilla cells (which are believed to be a control tower for hair follicle growth) were cultured in the presence of FGF18.

The present inventors have achieved the present invention based on this unexpected, novel finding that FGF18 (which was believed to be a hair growth promoting substance) “acts to inhibit hair growth” when administered continuously.

Considering that FGF18 is an endogenous factor present in hair follicles by nature, a substance that has an activity similar to that of FGF18 on hair follicles, a substance that promotes the activity of FGF18 per se or a substance that activates the expression of FGF18 can clearly be used as a hair growth-inhibiting agent or hair removing agent.

Substances which activate the expression of FGF18 may be screened for by monitoring whether a test substance promotes the expression of FGF18 or not in a cultured animal cell or an experimental animal.

Substances which promote the activity of FGF18 per se may be screened for by bringing a test substance together with FGF18 into contact with an FGF receptor on cell surfaces and monitoring whether the test substance promotes the activity of FGF18 per se on the FGF receptor.

Specifically, those substances may be obtained by a screening method comprising the following steps (a) to (c):

(a) compulsively expressing at least one FGF receptor gene selected from FGFR1c, FGFR2c, FGFR3c and FGFR4 on the surface of a cell by means of genetic engineering and culturing the cell,

(b) bringing, together with FGF18, a test substance into contact with the cell system obtained in step (a) having the FGF receptor on cell surfaces; and

(c) selecting those test substances which exhibited a higher cell growth promoting activity in step (b) than when FGF18 was allowed to act singly.

Further, on the assumption that a substance having an activity similar to that of FGF18 would have a similar hair growth-inhibiting effect, the present inventors focused on the reactivity of FGF18 on FGF receptors present in hair follicle cells. Since FGF18 reacts with FGFR1c, FGFR2c, FGFR3c and FGFR4 among the FGF receptor subclasses, a screening system for FGF18-like active substances was developed based on the above reactivity (Non-Patent Document 20).

As a result of extensive searches into substances derived, from naturally occurring plants and other natural products using FGF4 receptor as the screening system, several natural product-derived substances which activate FGF4 receptor were found. Thus, the present invention relating to FGF18-like active substances has also been achieved.

The present invention includes the following inventions.

(1) A hair growth-inhibiting agent or a hair removing agent both comprising FGF18 and/or an FGF18-like active substance and/or a substance that promotes the activity or expression of FGF18 as an active ingredient(s).
(2) The hair growth-inhibiting agent or hair removing agent according to (1), which is administered so that the FGF18 and/or FGF18-like active substance and/or substance that promotes the activity or expression of FGF18 is allowed to persist in hair follicles continuously.
(3) A hair growth-inhibiting agent or hair removing agent comprising as an active ingredient an expression vector carrying an FGF18-encoding DNA integrated thereinto, said agent being administered so that the FGF18 is expressed in hair follicles continuously.
(4) The hair growth-inhibiting agent or hair removing agent according to any one of (1) to (3), wherein said FGF18 is a full-length peptide of the amino acid sequence as shown in SEQ ID NO: 2 or a partial peptide there of having FGF18-like activity.
(5) The hair growth-inhibiting agent or hair removing agent according to (1) or (2), wherein the FGF18-like active substance is a Digenea simplex extract.
(6) The hair growth-inhibiting agent or hair removing agent according to any one of (1) to (5), which further comprises another hair growth-inhibiting agent or hair removing agent.
(7) A method of screening for the FGF18-like active substance according to (1) or (2) to thereby obtain candidates for the hair growth-inhibiting agent or hair removing agent from test substances, said method comprising the following steps (a) to (c):

(a) compulsively expressing at least one FGF receptor gene selected from FGFR1c, FGFR2c, FGFR3c and FGFR4 on the surface of a cell by means of genetic engineering and culturing the cell,

(b) bringing a test substance into contact with the cell system obtained in step (a) having the FGF receptor on cell surfaces; and

(c) selecting those test substances which exhibited FGF18-like cell growth promoting activity in step (b).

(8) A method of screening for the FGF18-like active substance according to (1) or (2) to thereby obtain candidates for the hair growth-inhibiting agent or hair removing agent from test substances, said method comprising the following steps (a) to (c):

(a) compulsively expressing four FGF receptor genes, FGFR1c, FGFR2c, FGFR3c and FGFR4, on respective cell surfaces by means of genetic engineering and culturing the four cells,

(b) bringing a test substance into contact with the four cell systems obtained in step (a) having the FGF receptors on cell surfaces; and

(c) selecting those test substances which, similar to EGF18, exhibit markedly higher cell growth promoting activity on the FGFR3c-expressing cell and the FGFR4-expressing cell than on the FGFR1c-expressing cell and the FGFR2c-expressing cell at the same concentration.

(9) The method according to (7) or (8), wherein the cell on whose surface the FGF receptor is compulsively expressed is mouse IL-3-dependent Ba/F3 cell strain.
(10) A method of screening for the substance that promotes the activity of FGF18 according to (1) or (2) to thereby obtain candidates for the hair growth-inhibiting agent or hair removing agent from test substances, said method comprising the following steps (a) to (c):

(a) compulsively expressing at least one FGF receptor gene selected from FGFR1c, FGFR2c, FGFR3c and FGFR4 on the surface of a cell by means of genetic engineering and culturing the cell,

(b) bringing, together with FGF18, a test substance into contact with the cell system obtained in step (a) having the FGF receptor on cell surfaces; and

(c) selecting those cell systems which exhibited a higher cell growth promoting activity in step (b) than when FGF18 was allowed to act singly,

(11) The method according to (10), wherein the FGF receptor is FGFR3c.
(12) The method according to (10), wherein the FGF receptor is FGFR4.
(13) The method according to any one of (10) to (12), wherein the cell on whose surface the FOP receptor is compulsively expressed is mouse IL-3-dependent Ba/F3 cell strain.
(14) A method of screening for the substance that promotes the activity of FGF18 according to (1) or (2) to thereby obtain candidates for the hair growth-inhibiting agent or hair removing agent from test substances, said method comprising the following steps (a) to (d):

(a) preparing an experimental animal or a cultured animal cell capable of expressing FGF18 to an observable extent,

(b) bringing a test substance into contact with or administering the same to the experimental animal or bringing the same into contact with the cultured animal cell,

(c) monitoring the expression of FGF18 in the experimental animal or the cultured animal cell after step (b), and

(d) selecting those test substances which have a function of promoting the expression of FGF18.

(15) The method according to (14), wherein the expression of FGF18 is monitored in step (c) by extracting mRNA from the experimental animal or the cultured animal cell after step (b) and then analyzing the mRNA level of expressed FGF18; and those test substances which have a function of promoting the expression of FGF18 are selected in step (d) by selecting systems that exhibited higher levels of FGF18 mRNA than when FGF18 was not allowed to act.

### EFFECT OF THE INVENTION

According to the present invention, it is possible to provide a hair growth-inhibiting agent or a hair removing agent by using or imitating an endogenous factor which inhibits the growth of hair follicles.

Further, according to the screening method of the present invention, it is possible to screen for substances which are effective as hair growth-inhibiting agent or hair removing agent.

### BRIEF DESCRIPTION OF THE DRAWINGS

FIG. 1. is a graph showing that the mRNA copy number of a hair follicle growth factor released by dermal papilla cells is suppressed at a low level when FGF18 is present continuously.

FIG. 2 is a diagram demonstrating in viva that it is possible to inhibit the growth of hair follicles by continuous administration of FGF18. In this Figure, A is a photomicrograph of a skin section from a control mouse that received PBS; and B is a photomicrograph of a skin section from a mouse that received FGF18.

FIG. 3 is a graph demonstrating that N-terminal truncated FGF18 partial peptides have an FGF receptor stimulating effect. (DNA synthesis levels when FGF receptor expressing cells are stimulated with about 100 ng/ml of samples are shown.) In this Figure, R1c, R2c, R1c and R4 represent FGFR1c expressing cell, FGFR2c expressing cell, FGFR3c expressing cell and FGFR4 expressing cell, respectively. Columns 1, 9, 16 and 26 are control columns without addition of samples; columns 2, 10, 17 and 27 received sample (d4); columns 3, 18 and 28 received sample (d12); columns 19 and 29 received sample (d16); columns 4, 20 and 30 received sample (d18); columns 5, 11, 21 and 31 received sample (d22); columns 6, 22 and 32 received sample (d37); column 12 received sample (d48); columns 13, 23 and 33 received sample (d67); columns 7, 14, 24 and 34 received sample (d77); and columns 8, 15 and 95 received sample (d95).

FIG. 4 is graphs demonstrating that C-terminal truncated FGF18 partial peptides have an FGF receptor stimulating effect. (DNA synthesis levels when FGF receptor expressing cells are stimulated with various concentrations of samples are shown.)

FIG. 5 R4/Ba/F3 Cell Growth Promoting Effect of Digenea simplex Extract Proliferation rates of R4/Ba/F3 cells when Digenea simplex extract was added at 0.083%, 0.83% and 8.3% in the presence and absence of FGF18 are shown.

FIG. 6 In vivo Analysis of Digenea simplex Extract

The time course of the state of hair regrowth in mouse dorsal skin is shown after Digenea simplex extract was administered twice subcutaneously to a dorsal site of C3H/HeN mouse in telogen phase of the hair cycle.

### BEST MODE FOR CARRYING OUT THE INVENTION

Hereinbelow, the present invention will be described in detail.

By applying the present invention, it is possible to provide a novel hair growth-inhibiting agent and hair removing agent. These hair growth-inhibiting agent and hair removing agent share the common feature of utilizing the effect that FGF18 of consistently high concentrations inhibits the growth of hair follicles (sometimes called “hair roots”).

The hair follicle is an organ that produces hair. The hair follicle growth cycle consists of a growth phase (anagen), a recessing phase (catagen) and a resting phase (telogen) which follows the recessing phase. After the resting phase, the growth phase begins. Generally, in mouse experimental systems, anagen phase is a period from day 1 to day 19 after depilation; and catagen phase is a period from day 20 to day 21 after depilation. It is also known that the hair follicle cycle enters telogen phase at day 21-22 after depilation. During the anagen phase, the growth (elongation) of new hair is activated. Simultaneously, the growth of hair follicles is activated inside the skin, and the bottom part of hair follicles reaches the vicinity of the skin bottom. On the other hand, during the telogen phase, hair follicles stay shallow in the skin in small sizes. Further, the thickness of the skin is completely different between anagen phase and telogen phase. In mice with a colored body hair, melanin is synthesized at the beginning of the anagen phase and a blue skin is visible. Therefore, it is also possible to evaluate the progress of hair follicle growth cycle by observing the blue color from the outside of the skin. Further, when the skin is dissected during the anagen phase and observed from the reverse side, the reverse side of the skin can be seen black because hair follicles with abundant melanin are standing in lines at a high density. To the contrary, during the telogen phase, the reverse side of the skin can be seen remaining white. For example, in mouse experimental systems, the entire dorsal hair of 7 to 8 week-old mice is in telogen phase; by depilating the grown hair, the hair cycle is synchronized and anagen phase begins.

### 1. FGF18

FGF18 is synthesized in the cytoplasm of FGF18 producing cells as a polypeptide of 207 amino acids in human and mouse. When this polypeptide is secreted to the outside of cells, its N-terminal signal peptide is cleaved to generate a secreted polypeptide of 181 amino acids which exerts a physiological action. It has been confirmed that FGF18 reacts with at least four out of the seven FGF receptor subclasses (FGFR1c, FGFR1b, FGFR2c, FGFR2b, FGFR3c, FGFR3b and FGFR4) and they are FGFR1c, FGFR2c, FGFR3c and FGFR4 (Non-Patent Document 20).

Examples of the FGF18 contained in the novel hair growth-promoting agent, hair regrowth-promoting agent or therapeutic for alopecia of the present invention include a human-derived FGF18 consisting of the amino acid sequence as shown in SEQ ID NO: 2. FGF18 polypeptides which may be used in the present invention are not limited to human-derived polypeptides but include, for example, polypeptides derived from other mammals. Specific examples of other mammals include, but are not limited to, mouse, rat, chicken, turkey, cattle, pig, sheep and rabbit. For example, it is possible to isolate a gene encoding FGF18 from a non-human mammal by designing a probe based on the nucleotide sequence of a human-derived FGF18 as shown in SEQ ID NO: 1 and using the probe according to conventional methods.

The nucleotide sequence and the amino acid sequence of secretion signal sequence-deleted human-derived FGF18 are shown in SEQ ID NOS: 1 and 2, respectively. The nucleotide sequence and the amino acid sequence of secretion signal sequence-deleted mouse-derived FGF18 are shown in SEQ ID NOS: 3 and 14, respectively. As comparison of the amino acid sequences as shown in SEQ ID NOS: 2 and 14 reveals, FGF18 polypeptides have a very high homology in mammals. Thus, it is understood that the function of FGF18 is almost the same among mammals.

In the present invention, “the number of amino acids deleted at the N-terminal” means the number of deleted amino acids excluding the methionine residue introduced for initiation of translation in recombinant protein production. Those proteins which consist of the amino acid sequence as shown in SEQ ID NO: 2 or 14, with one or several amino acids being deleted, substituted or added, and which have a hair follicle growth-inhibiting effect are included in the FGF18 of the present invention. It should be noted that “several amino acids” means, for example, 2 to 37 amino acids, preferably 2 to 22 amino acids, more preferably 2 to 18 amino acids, even more preferably 2 to 12 amino acids, and most preferably 2 to 4 amino acids.

The region spanning from positions 32 to 151 of the amino acid sequence as shown in SEQ ID NO: 14 is believed to be a core domain common to various FGF18 polypeptides; it has been observed that this domain binds to receptors and heparin (Non-Patent Document 1). Therefore, partial peptides comprising positions 32 to 151 of the amino acid sequence as shown in SEQ ID NO: 14 or mutant peptides with one or several amino acids deleted, substituted or added in a region other than the above-described region from positions 32 to 151 may be used as the FGF18 of the present invention as long as those peptides exert the hair follicle growth-inhibiting effect possessed by FGF18.

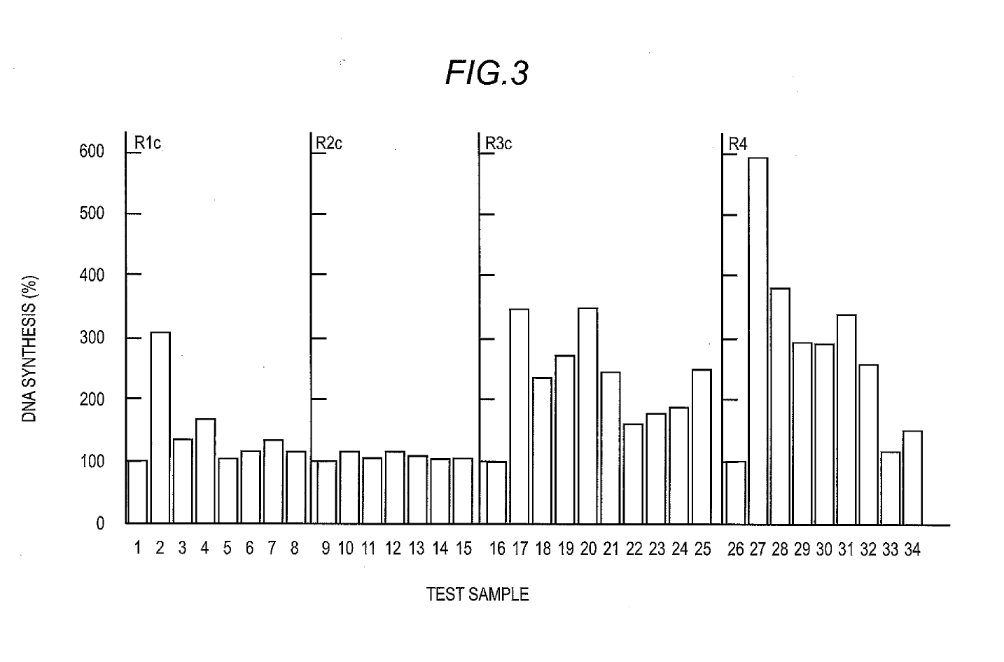

Actually, according to the results of experiments (FIG. 3) where the effect on FGF18 activity of deleting 4 to 95 amino acids (excluding the methionine residue) from the N-terminal of SEQ ID NO: 14 (d4-d95) was determined, even d95 possesses FGF18-like FGFR3c stimulating activity although it is weak.

Therefore, the amino acids at positions 1 to 95 may be entirely deleted from the N-terminus of SEQ ID NO: 14 (excluding the methionine residue) and yet the resulting polypeptide can be used as FGF18 in the present invention. Preferably, a partial peptide with the amino acids at positions 1 to 77 deleted from the N-terminus is used. More preferably a partial peptide with the amino acids at positions 1 to 37 deleted from the N-terminus, still more preferably a partial peptide with the amino acids at positions 1 to 22 deleted from the N-terminus, even more preferably a partial peptide with the amino acids at positions 1 to 12 deleted from the N-terminus, and most preferably a partial peptide with the amino acids at positions 1 to 4 deleted from the N-terminus are used. Further, a part of the N-terminal sequence from positions 1 to 95 may be substituted; preferably, a partial peptide comprising the amino acid sequence from positions 24 to 182 of SEQ ID NO: 14 may be used; and more preferably, a partial peptide comprising the amino acid sequence from positions 6 to 182 of SEQ ID NO: 14 may be used.

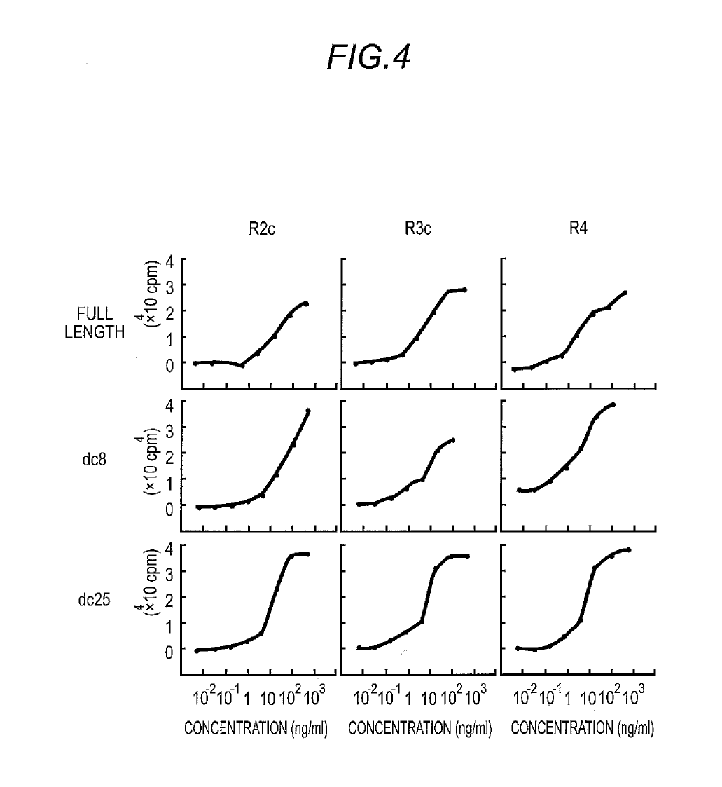

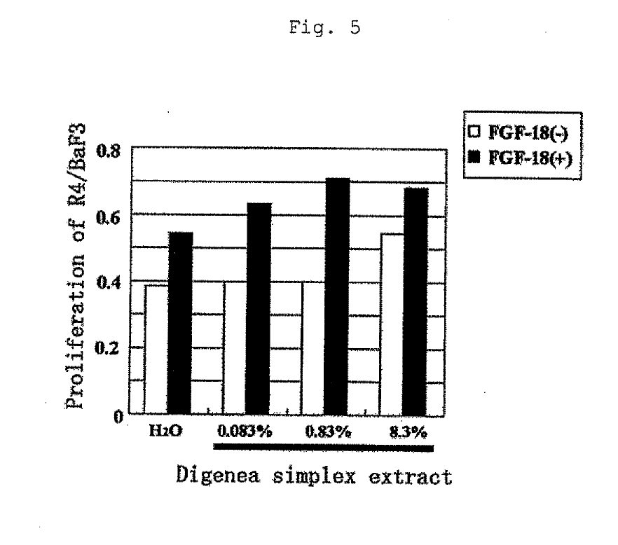

With respect to the C-terminal side, according to the results of experiments where the effect on FGF18 activity of deleting C-terminal 8 to 25 amino acids of SEQ ID NO: 14 (dc8-dc25) was determined, even dc25 possesses FGF18-like FGFR2c, FGFR3c and FGFR4 stimulating activity strongly (FIG. 4). Therefore, the amino acids at positions 1 to 25 may be entirely deleted from the C-terminus of SEQ ID NO: 14 and yet the resulting polypeptide can be used as FGF18 in the present invention.

As described above, the term “FGF18” as used herein includes not only full-length polypeptides of FGF18 derived from mammals such as human and mouse, but also FGF18 partial peptides and FGF18 mutant peptides having FGF18-like activity.

### 2. FGF18-Like Active Substances, FGF18 Activating Substances and Screening Methods for Such Substances

### [1] FGF18-Like Active Substances

The term “FGF18-like active substance” means a substance which binds to a receptor similar to the receptor that FGF18 binds to and which activates an intracellular signal transduction system similar to the system that FGF18 activates. Since FGF18 reacts with at least the four FGF receptor subclasses FGFR1c, FGFR2c, FGFR3c and FGFR4 among the seven FGF receptor subclasses as described above, the “FGF18-like active substance” of the present invention means a substance which binds to any of the receptors FGFR1c, FGFR2c, FGFR3c and FGFR4 on the surface of a cell and activates the intracellular signal transduction system of the cell in the same manner as FGF18. (However, among those FGF18-like active substances, FGF18 partial peptides and FGF18 mutant peptides are included in the “FGF18” in the present invention.) In order to specifically examine whether or not a substance activates the intracellular signal transduction system of a cell that possesses FOP receptor on its surface, the amounts of intracellular second messengers (such as Ca ion or cAMP) may be measured. Typically, this activation is confirmed when proliferation of an FGF receptor-expressing cell has been observed after administering the substance to the cell or transferring the gene of the substance into the cell and expressing the gene therein.

In particular, FGF18 has a strong effect to activate the intracellular signal transduction system in FGFR3c-expressing cell and FGFR4-expressing cell among receptor-expressing cells. Therefore, it is also possible to select FGF18-like active substances as substances having a strong activity upon FGFR3c-expressing cell or FGFR4-expressing cell. Further, it is also possible to select FGF18-like active substances as substances whose cell growth promoting activity upon FGFR3c-expressing cell and FGFR4-expressing cell is markedly higher (at least twice, preferably at least three times) than their cell growth promoting activity upon FGFR1c-expressing cell and FGFR2c-expressing cell at the same concentration.

### [2] Screening Methods for FGF18-Like Active Substances

Screening for those substances which potentially have a function of hair growth inhibition, hair removal or the like can be performed by examining whether or not a test substance has the above-described FGF18-like activity.

Specifically, a screening method comprising the following steps (a) to (c) may be used.

(a) compulsively expressing at least one FOP receptor gene selected from FGFR1c, FGFR2c, FGFR3c and FGFR4 on the surface of a cell by means of genetic engineering and culturing the cell,

(b) bringing a test substance into contact with the cell system obtained in step (a) having the FOP receptor on cell surfaces; and

(c) selecting those test substances which exhibited FGF18-like cell growth promoting activity in step (b).

Alternatively, a screening method comprising the following steps (a′) to (c′) may be used.

(a′) compulsively expressing four FGF receptor genes, FGFR1c, FGFR2c, FGFR3c and FGFR4, on respective cell surfaces by means of genetic engineering and culturing the four cells,

(b′) bringing a test substance into contact with the four cell systems obtained in step (a′) having the FOP receptors on cell surfaces; and

(c′) selecting those test substances which, similar to EGF18, exhibit markedly higher cell growth promoting activity on the FGFR3c-expressing cell and the FGFR4-expressing cell than on the FGFR1c-expressing cell and the FGFR2c-expressing cell at the same concentration.

As the FGF receptor gene, at least one of the FOP receptor genes FGFR1c, FGFR2c, FGFR3c and FGFR4 is used; it has been confirmed that FGF18 binds to these receptors and that FGF18 exerts cell growth effect upon cells having these receptors on their surfaces. Preferably, FGFR3c or FGFR4 is used. To bring a test substance into contact with a cell, it is typically directly added to the cell culture broth, but in a particular case where the test substance is a protein, a gene encoding the test substance can be transferred into an FGF receptor-expressing cell.

The cell to be used for compulsive expression of an FGF receptor on its surface may be any cell as long as it can be cultured. Preferably, mouse IL-3-dependent Ba/F3 cell strain is used.

The parent cell with no FOP receptor gene transferred thereinto may be used in a control test. It is preferable to provide a step of performing the similar operation as in step (b) using a test substance to confirm that the test substance does not cause cell growth promoting activity in such parent cells.

[3] Substances that Promote the Activity of FGF18

Substances even if they can not be said to be FGF18-like active substance as defined in [1] above, may foster the hair follicle growth-inhibiting effect of FGF18 by, for example, promoting or enhancing the binding of FGF18 to receptors; since these substances have hair growth-inhibiting effect or hair removing effect, they are referred to as “substances that promote the activity of FGF18” in the present invention. These substances may be used either alone or in combination with FGF18, etc. as a hair growth-inhibiting agent or hair removing agent.

[4] Screening Method for Substances that Promote the Activity of FGF18 Substances that promote the activity of FGF18 may be screened for by bringing, together with FGF18, a test substance into contact with cell surface FGF receptors and then monitoring whether the activity of FGF18 per se on FGF receptors has been promoted.

Specifically, substances that promote the activity of FGF18 may be obtained by a screening method comprising the following steps (a) to (c):

(a) compulsively expressing at least one FGF receptor gene selected from FGFR1c, FGFR2c, FGFR3c and FGFR4 on the surface of a cell by means of genetic engineering and culturing the cell,

(b) bringing, together with FGF18, a test substance into contact with the cell system obtained in step (a) having the FGF receptor on cell surfaces; and

(c) selecting those cell systems which exhibited a higher cell growth promoting activity in step (b) than when FGF18 alone was allowed to act singly.

Those test substances which increased the cell growth promoting activity of FGF18 on cells expressing FGF receptors (such as FGFR3c or FGFR4) in this screening method are believed to be substances that promote the activity of FGF18 in hair follicle cells, so they can be judged to have a potential function of hair growth inhibition or hair removal.

Accordingly, the FGF18 activity promoting substance screened through those steps is a promising candidate for a hair growth-inhibiting agent or hair removing agent. As a criterion for screening, those substances which increased the proliferation capacity of FGF receptor expressing cells by 5% or more, preferably by 10% or more, more preferably by 20% or more, may be selected.

3. Substances that Promote the Expression of FGF18 and Screening Method for Such Substances

Since FGF18 is an endogenous factor present in hair follicles, a substance that promotes the transcription and translation of FGF18 in hair follicle cells (i.e., substance that activates the expression of FGF18) is also capable of increasing the concentration of FGF18 in hair follicle cells to thereby induce the hair growth inhibiting activity of FGF18. Thus, such a substance has a hair growth inhibiting or hair removing effect.

Such substances that activate the expression of FGF18 may be screened for by monitoring whether or not a test substance promotes the expression of FGF18 in cultured animal cells (such as skin-derived cells) or experimental animals.

Specifically, first, a test substance is brought into contact with or administered to an experimental animal, or a test substance is brought into contact with a cultured animal cell. Experimental animals refer to non-human animals such as mouse, rat, chicken, turkey, cattle, pig, sheep and rabbit. Examples of test substances include, but are not limited to, plant extracts, peptides, proteins, nonpeptidic compounds, low molecular weight compounds, synthetic compounds, fermentation products, cell extracts and animal tissue extracts. These substances may be either novel substances or known substances. To bring it into contact with a cell or animal, the test substance is typically directly added to the cell culture broth or administered to the experimental animal but in a particular case where the test substance is a protein, a gene encoding the test substance can be transferred into an FGF receptor-expressing cell.

Subsequently, the expression of FGF18 in the cultured animal cell or experimental animal is monitored. The expression of FGF18 in the cultured animal cell or experimental animal may be monitored, for example, by analysis with conventional methods such as ELISA using. FGF18 antibody or by analyzing the FGF18 mRNA level in the cell or experimental animal by quantitative reverse transcription PCR or Northern blotting.

If as a result of analysis by any of the above-listed methods, the expression level of FGF18 in the cultured animal cell or experimental animal cultured in the presence of a test substance is found to be greater than that level in the animal cell cultured in the absence of the test substance, the test substance may be judged to have a potential function of hair growth inhibition or hair removal. Specifically, if the mRNA level is at least 20%, preferably at least 50%, more preferably at least 80%, still more preferably at least 100% greater than in the case where the test substance was not allowed to act, the latter can positively be regarded as a substance that promotes the expression of FGF18. The expression levels of FGF18 mRNA in cultured keratinocytes, cultured dermal cells or cultured dermal papilla cells vary considerably depending on culture conditions or the type of the cells, so they may be measured individually by the above-mentioned methods or the like and 1.2-fold or greater increases in measured values may be used as a criterion for screening.

The FGF18 expression promoting substance screened through the above-described steps may be used either alone or in combination with FGF18, etc. as a hair growth-inhibiting agent or a hair removing agent.

### 4. Hair Growth Inhibiting Agent and Hair Removing Agent

In the present invention, it is necessary to administer the above-described FGF18, FGF18-like active substance, FGF18 activating substance or FGF18 expression promoting substance (hereinafter, referred to as “FGF18 or the like”) in such a manner that FGF18 or the like is allowed to persist in hair follicles continuously. The expression “persist continuously” means such a state that FGF18 or the like remains in hair follicles at a concentration of at least 1%, preferably at least 5%, more preferably at least 10% of the initial concentration, for a period of at least 1 day, preferably 4 days, more preferably 8 days.

In order to achieve such a state, a method may be used in which FGF18 or the like is gradually absorbed from the skin using implants or patches. Alternatively, FGF18 or the like may be administered repeatedly depending on necessity, for example, at intervals of 1 to 3 days or at least twice a day.

With respect to specific dosage forms, FGF18 or the like is formulated either alone or in combination into preparations adapted for skin application (e.g., solutions, creams, ointments, gels, lotions, shampoos, aerosols or patches) and is supplied as a hair growth inhibiting agent or a hair removing agent.

In particular, the hair growth inhibiting agent or hair removing agent is administered in the form of a pharmaceutical composition comprising FGF18 or the like together with a pharmacologically acceptable carrier adapted for local application. The hair growth inhibiting agent or hair removing agent comprising FGF18 or the like contains an active compound in a pharmacologically acceptable carrier usually at about 0.01 to about 100 μg/day/cm², preferably about 0.1 to about 10 μg/day/cm². This means that the concentration of FGF18 or the like is usually about 0.01 to about 100 μg/day/cm², preferably about 0.1 to about 10 μg/day/cm² in a pharmacologically acceptable carrier.

Further, the hair growth inhibiting agent or hair removing agent may comprise other hair growth inhibiting agents or hair removing agents well known in the art. Substances which increase or enhance the hair growth inhibiting or hair removing effect of FGF18 or the like are not particularly limited.

Further, the hair growth inhibiting agent or hair removing agent may comprise compounds or drugs well known in the art that exhibit hair growth inhibiting activity or hair removing activity. Other hair growth inhibiting agents or hair removing agents are not particularly limited.

Further, the pharmacologically acceptable carrier adapted for local application is not particularly limited. Specific examples include, but are not limited to, ointments such as hydrophilic vaseline or polyethylene glycol ointment; pastes such as gum (e.g., xanthan gum); solutions such as alcoholic, aqueous or buffer solution; gels such as aluminum hydroxide or sodium alginate gel; albumins such as human or animal albumin; collagens such as human or animal collagen; celluloses such as alkyl cellulose, hydroxyalkyl cellulose or alkylhydroxyalkyl cellulose; methyl cellulose, hydroxyethyl cellulose, carboxymethyl cellulose, hydroxypropylmethyl cellulose and hydroxypropyl cellulose; polymers such as Pluronic™ polyol as exemplified by Pluronic F-127; tetronics such as Tetronic 1508; and alginates such as sodium alginate.

The hair growth inhibiting agent or hair removing agent of the present invention may be one that comprises, as an active ingredient, an expression vector carrying an FGF18-encoding DNA as described in the above subsection “1. FGF18” (i.e., a DNA encoding the full-length polypeptide of FGF18 derived from a mammal such as human, or a partial peptide thereof or a mutant peptide thereof both having FGF18-like activity). This means that the hair growth inhibiting agent or hair removing agent of the present invention may be used in gene therapy using the above expression vector. The expression vector is not particularly limited, although it has sequences (such as promoter) to drive the expression of FGF18 or the like in animal cells. Examples of useful expression vectors include, but are not limited to, plasmid vectors and virus vectors.

More specifically, in gene therapy, the FGF18-encoding DNA as described in the above subsection “1. FGF18” is integrated in, for example, a virus vector. Then, an avirulent virus comprising the resultant recombinant virus vector is transfected into patients. FGF18 is produced in patients' bodies. This FGF18 is capable of inhibiting the growth of hair follicles.

As a method of introducing the hair growth inhibiting agent or hair removing agent for gene therapy into cells, both a gene transfer method using a virus vector and a non-viral gene transfer method [Nikkei Science, 1994 April issue, pp. 20-45; Experimental Medicine Extra Issue 12(15)(1994); Experimental Medicine Separate Volume “Basic Techniques for Gene Therapy”, Yodo-sha (1996)] may be used.

As examples of gene transfer method using a virus vector, methods may be mentioned in which a DNA encoding TR4 or mutant TR4 is integrated in DNA or RNA viruses such as retrovirus, adenovirus, adeno-associated virus, herpes virus, vaccinia virus, poxvirus, poliovirus and sinbis virus. Among these, methods using retrovirus, adenovirus, adeno-associated virus or vaccinia virus are especially preferred.

Examples of non-viral gene-transfer methods include a method in which an expression plasmid is directly administered into muscle (DNA vaccine method), the liposome method, lipofection, microinjection, the calcium phosphate method, electroporation and so on. The DNA vaccine method and the liposome method are especially preferred.

In order to allow the hair growth inhibiting agent or hair removing agent for gene therapy to actually work as a pharmaceutical, an in vivo method (in which DNA is introduced into the body directly) or an ex vivo method (in which a certain type of cell is taken out of the human body; DNA is introduced into the cell ex vivo; and then the cell is returned to the body) [Nikkei Science, 1994 April issue, pp. 20-45; The Pharmaceuticals Monthly; 36(1), 23-48 (1994); Experimental Medicine Extra Issue 12(15) (1994)] may be used.

For example, when the agent for gene therapy is administered by an in vivo method, it may be administered through an appropriate route, such as intravenous, intra-arterial, subcutaneous, intradermal or intramuscular route, depending on the type of disease and the severity of condition. When the agent for gene therapy is administered by an in vivo method, the agent is generally administered in the form of injections to which conventional carriers may be added, if necessary. Alternatively, when the agent for gene therapy is administered in the form of liposomes or membrane-fused liposomes (e.g., Sendai virus-liposome), liposome preparations such as suspensions, frozen preparations, centrifuged/concentrated/frozen preparations, or the like may be used.

### 5. Digenea Simplex Extract

One of the FGF18-like active substances obtained by the screening method described in the above subsection 2. [2] is Digenea simplex extract. This extract has been confirmed to have a hair regrowth inhibiting effect.

The raw material Digenea simplex belongs to the family Rhodomelaceae, the order Ceramiales in the class Florideophyceae. It is also called “Makuri”.

In obtaining extract from Digenea simplex, the entire part of this plant may be used; individual parts may be used either independently or in an appropriate combination.

Further, the plant may be used whether it is in a dry or non-dry state.

For obtaining an extract to be used in the present invention from Digenea simplex, the raw material may be shredded or crushed and then extracted with an appropriate solvent by conventional extraction methods. The solvent used is not particularly limited. For example, water or anhydrous or hydrous organic solvents may be enumerated. Examples of anhydrous or hydrous organic solvents include one or more substances selected from the group consisting of monohydric alcohols, polyhydric alcohols or derivatives thereof ketones, esters, ethers, petroleum ether, aliphatic hydrocarbons, halogen compounds and aromatic hydrocarbons. Specific examples of the solvent include, but are not limited to, water, methanol, ethanol, butanol, acetone and ethyl acetate ester. They may be used either alone or in combination. Among these, use of water or a monohydric alcohol such as methanol or ethanol is especially preferable. For extracting purposes, the amounts of the above-listed solvents are not particularly limited. The amount of the solvent may be 0.1-1000 times, preferably 1-100 times, more preferably 2-50 times by weight the amount of the raw material Digenea simplex.

The extraction method using the above-listed solvents may be performed according to conventional procedures. For example, as regards the extraction temperature, extraction may be performed at around room temperature or at a temperature around the boiling point of the solvent used. As regards the extraction operation, a dried and crushed or a simply crushed Digenea simplex may be soaked in a solvent at room temperature for 1-30 days or may be extracted under reflux at a temperature around the boiling point of the solvent.

As will be described later in Example 4, the Digenea simplex extract was searched for as one of those substances which have FGF18-like cell growth promoting activity by the screening method of the present invention using a cell upon whose surface FGFR4 (an FGF receptor) was compulsively expressed by means of genetic engineering. This Digenea simplex extract exhibited a similar cell growth promoting activity to FGF18 on FGFR4-expressing cells, but exhibited no cell growth promoting activity on the parent cell strain which does not express FGFR4. Thus, the Digenea simplex extract can be said to be a FGF18-like active substance.

Further, as expected, this FGF18-like active substance was demonstrated to have a hair regrowth inhibiting effect (see Example 6).

With respect to the cell growth promoting effect on FGFR4-expressing cells, an experiment was performed to compare the effect of using FGF18 alone with that of using FGF18 together with Digenea simplex extract. The results confirmed that low concentrations of Digenea simplex extract promote the activity of FGF18. Therefore, Digenea simplex extract can be said to be a substance that promotes the activity of FGF18. From these results, it can be seen that the technique of this comparative experiment may be used as a screening method for substances that promote the activity of FGF18. It is evident that this technique is a promising means for selecting candidates for hair regrowth inhibiting agents.

### EXAMPLES

Hereinbelow, the present invention will be described with reference to the following Examples. However, the technical scope of the present invention is not limited to these Examples.

### Example 1

### Effect of FGF18 on Gene Expression in Dermal Papilla Cells

In this Example, in order to evaluate the function of FGF18 on hair growth, the effect of FGF18 on gene expression was examined in dermal papilla cells which are believed to be a control tower for hair follicle growth.

### Materials and Methods

Cultured human dermal papilla cells (HFDP; from adult human scalp; Toyobo) were subcultured in HFDP growth medium (20% fetal bovine serum-containing basal medium for dermal papilla cell; Toyobo) and used in experiments within two passages. The cells were treated with trypsin and seeded in 24-well collagen-coated plates (Sumilon) at a density of 1×10⁴ cells/well. The cells were maintained at 37° C. in HFDP medium. On the next day, FGF18 was added to the medium. After the cells were cultured therein for 24 hr, the medium was exchanged for FGF18 free medium. Control group was cultured in FGF18 free medium. Subsequently, the cells were harvested and the mRNA was extracted and purified. The expression level of mRNA from VEGF (known as a hair follicle growth promoting factor) contained in the resultant mRNA was analyzed.

### <Results>

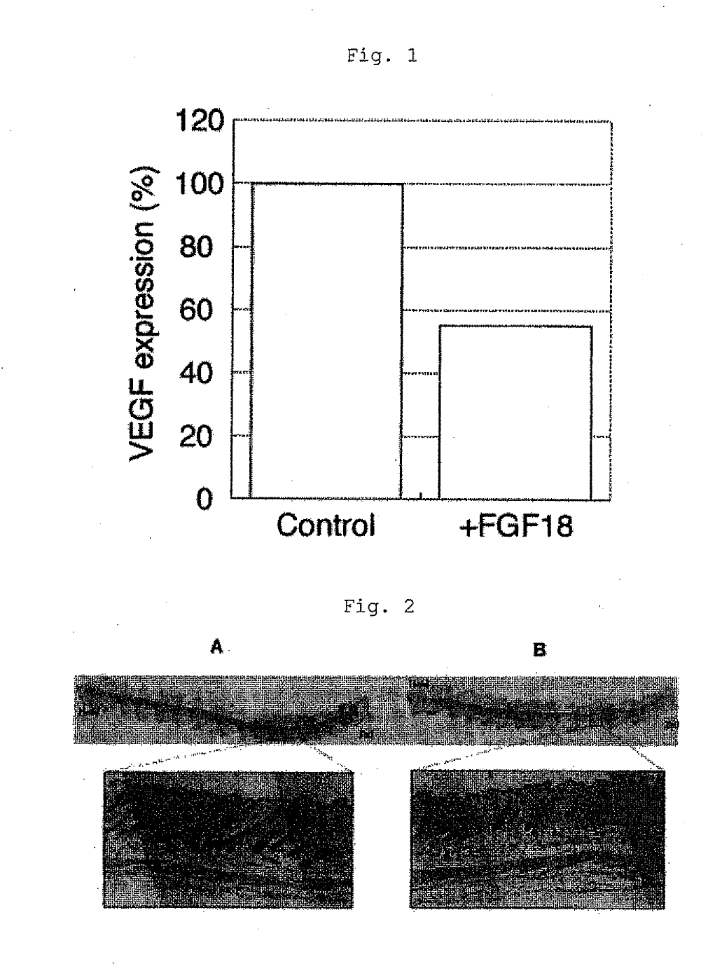

The results are shown in FIG. 1. It is believed that dermal papilla cells release various factors to thereby support hair growth. On of such factors is VEGF. In this experiment, addition of FGF18 to the medium decreased the expression level of VEGF mRNA in dermal papilla cells. Therefore, it was confirmed that FGF18 inhibits the production of one factor for hair growth that is released by dermal papilla cells.

### Example 2

### Inhibitory Effect of FGF18 on Hair Growth

In this Example, in order to examine the effect of FGF18 in vivo on hair follicle growth, the effect of administering GF18 was tested on C3H/HeN mice that had been induced to a hair follicle anagen phase.

### Materials and Methods

In order to examine the effect of FGF18 in vivo on hair follicle growth, FGF18 protein dissolved in phosphate buffered physiological saline (PBS) was administered to hair follicle anagen phase-induced mice.

Briefly, dorsal hair of 50 day-old C3H/HeN male mice in telogen phase was depilated gently with fingers to thereby induce the start of hair follicle anagen phase. Then, FGF solution was injected into the dorsal skin subcutaneously from the vicinity of the tail (1 μg of FGF18 per mouse). The mice were maintained on a diet and water ad libitum. Starting from this day, FGF18 solution was injected subcutaneously into the dorsal skin every day at about the same time for 8 days. The control group received injection of PBS instead of FGF18. Nine days after the initial injection, the mice were euthanized under anesthesia. Full thickness skin samples were excised from the dorsal part and embedded in paraffin. The thus embedded skin samples were sliced into 4 μm thick sections with a microtome, stained with hematoxylin and observed under microscope.

### <Results>

The results are shown in FIG. 2. In this Figure, A represents a microphotograph of a skin section from a control mouse which received PBS; B represents a microphotograph of a skin section from an FGF18-administered mouse. In A and B, the magnification is the same.

As seen from microphotograph A, natural growth of hair follicles was observed at day 9 in the PBS-administered mouse. Hair follicles had grown long and reached the lower layer of the skin.

On the other hand, in the FGF18 solution-administered mouse in microphotograph B, hair follicles are short, suggesting that hair growth is strongly inhibited.

From these results, it was demonstrated in vivo that continuous administration of FGF18 is capable of inhibiting the growth of hair follicles.

### Example 3

### FGF Receptor Stimulating Effect of FGF18 Partial Peptides

As FGF18 partial peptides, partial polypeptides were prepared based on SEQ ID NO: 14 which corresponds to the amino acid sequence of mouse FGF18. Briefly, amino acids from position 4 to position 95 as counted from N-terminus (excluding methionine) were deleted to prepare partial peptides d4-d95 [indicating the number of amino acids deleted from N-terminal (excluding methionine)]. As additional FGF18 partial peptides, partial polypeptides were prepared based on SEQ ID NO: 14 which corresponds to the amino acid sequence of mouse FGF18 by deleting amino acids from position 8 to position 25 as counted from C-terminus. Thus obtained were partial peptides dc8-dc25 [indicating the number of amino acids deleted from C-terminal].

The amino acid sequences (nucleotide sequences) of the individual partial peptides correspond to the following SEQ ID NOS.

d4: SEQ ID NO: 15 (SEQ ID NO: 4)
d12: SEQ ID NO: 16 (SEQ ID NO: 5)
d16: SEQ ID NO: 17 (SEQ ID NO: 6)
d18: SEQ ID NO: 18 (SEQ ID NO: 7)
d22: SEQ ID NO: 19 (SEQ ID NO: 8)
d37: SEQ ID NO: 20 (SEQ ID NO: 9)
d48: SEQ ID NO: 21 (SEQ ID NO: 10)
d67: SEQ ID NO: 22 (SEQ ID NO: 11)
d77: SEQ ID NO: 23 (SEQ ID NO: 12)
d95: SEQ ID NO: 24 (SEQ ID NO: 13)
dc8: SEQ ID NO: 28 (SEQ ID NO: 25)
dc15: SEQ ID NO: 29 (SEQ ID NO: 26)
dc25: SEQ ID NO: 30 (SEQ ID NO: 27)

FGF18 stimulates the four FGF receptors FGFR1c, FGFR2c, FGFR3c and FGFR4, but the intensity of stimulation varies depending on the receptor. It is believed that summation of stimulations on these receptors results in inhibition of hair growth.

According to teachings disclosed in literature, the FGF receptor genes FGFR1c, FGFR2c, FGFR3c and FGFR4 were respectively introduced by means of genetic engineering into Ba/F3 cell strain (obtained from RIKEN BRC) which is mouse IL-3-dependent proB cell to thereby prepare cells on whose surface FGFR was compulsively expressed (Ornitz, D M, Xu, J., Colvin, I S., McEwen, D G, MacArthur, C A., Coulier, R, Gao, G. and Goldfarb, M., 1996. Receptor specificity of the fibroblast growth factor family. I Biol. Chem. 271(25):15292-15297; Yoneda, A., Asada, M., Oda, Y, Suzuki, M. and Irnamura, T, 2000. Engineering of an FGF-proteoglycan fusion protein with heparin-independent, mitogenic activity. Nat. Biotec. 18(6):641-644).

Using these cells, the individual receptor stimulating activities were examined. The results are shown in FIG. 3.

Test results using FGFR1c expressing cell (R1c), FGFR2c expressing cell (R2c), FGFR3c expressing cell (R3c) and FGFR4 expressing cell (R4) are shown in this order in FIG. 3, with R1c on the left side.

The longitudinal axis represents the DNA synthesis level when cells were stimulated with about 100 ng/ml of a sample, taking the basal DNA synthesis level for no cell stimulation as 100%.

<R1c>
Column 1: no sample (control). Column 2: (d4). Column 3: (d12). Column 4: (d18).
Column 5: (d22). Column 6: (d37). Column 7: (d77). Column 8: (d95)
<R2c>
Column 9: no sample (control). Column 10: (d4). Column 11: (d22). Column 12: (d48).
Column 13: (d67). Column 14: (d77). Column 15: (d95).
<R3c>
Column 16: no sample (control). Column 17: (d4). Column 18: (d12). Column 19: (d16). Column 20: (d18). Column 21: (d22). Column 22: (d37). Column 23: (d67).
Column 24: (d77). Column 25: (d95).

### <R4>

Column 26: no sample (control). Column 27: (d4). Column 28: (d12). Column 29: (d16). Column 30: (d18). Column 31: (d22). Column 32: (d37). Column 33: (d67). Column 34: (d77).

FIG. 4 shows the test results using FGFR2c expressing cell (R2c), FGFR3c expressing cell (R3c) and FGFR4 expressing cell (R4) in this order, with R2c on the left side.

The longitudinal axis represents the DNA synthesis level in CPM when cells were stimulated with various concentrations of samples, taking the basal DNA synthesis level for no cell stimulation as 0 CPM. The horizontal axis represents the concentration of each sample used to stimulate cells.

<R2c>
Upper graph: full length (control). Center graph: (dc8). Lower graph: (dc25)
<R3e>
Upper graph: full length (control). Center graph: (dc8). Lower graph: (dc25)

### <R4>

Upper graph: full length (control). Center graph: (dc8). Lower graph: (dc25)

From FIG. 3 and FIG. 4, even FGF18 partial peptides (d4-d95, dc8-dc25) are recognized to have an activity of stimulating FGF receptors with various intensities and thereby stimulating the DNA synthesis by cells. In particular, FGF18 partial peptides were shown to have an activity of strongly stimulating FGFR3c expressing cell and FGFR4 expressing cell. These experimental results, showing that FGF18 partial peptides have an activity similar to the FGF receptor stimulating activity of FGF18, indicate that these partial peptides also have an FGF18-like activity in hair growth regulation.

Among the FGF18 partial peptides, d4-d37 and dc8-dc25, especially d4-d22 and dc8-dc25 exhibited strong receptor stimulating activity on both FGFR3c and FGFR4. Therefore, the partial peptides where up to 37 amino acids, especially up to 22 amino acids (excluding methionine) are deleted from the N-terminal side, and the partial peptide where up to 25 amino acids are deleted from the C-terminal side are preferably used as the FGF18 of the present invention.

### Example 4

### Screening for FGF18-Like Active Substances Using FGFR4 Expressing Cell

The cell on whose surface FGR4 is compulsively expressed (R4/Ba/F3 cell) prepared above was cultured in the presence of various plant extracts as test solutions. As positive control, a commercial FGF18 protein was used. The cell count after culturing for a specific time period was determined with Cell Counting Kit-8 (manufactured by Dojindo Laboratories and sold by Wako Pure Chemical Industries) by measuring the coloring at 450 nm which was proportionate to the yield of WST-8 formazan.

As a result, it was found that Digenea simplex extract acts as FGF18-like active substance and is capable of promoting the growth of R4/Ba/F3 cell.

### Example 5

### Cell Growth Promoting Activity of the FGF18-Like Active Substance of the Invention

To 2.0 g of dried Digenea simplex (Ryukyu Marine Product Processing Plant, or Nago Non Shokai), 40 ml of 70% ethanol aqueous solution was added. Then, soaking extraction was performed at room temperature for 7 days. To 2.0 g of dried Digenea simplex (Ryukyu Marine Product Processing Plant, or Nago Non Shokai), 40 ml of distilled water was added. The resultant mixture was boiled for 20 min. Crude extracts obtained from these procedures were filtered, and the filtrates were collected to give extracts.

In the same manner as in Example 3, the cell growth promoting activity of the FGF18-like active substance of the present invention was measured using R4/Ba/F3 cell. Specifically, measurement was performed as described below. Briefly, RPMI1640 medium containing 10% FBS and 1% Antibiotic G-418 Sulfate (Promega; V7983) was added to each well of 96-well cell culture plates (50 μl/well), Subsequently, various concentrations of test solutions prepared by diluting samples with water were added (10 μl/well), followed by addition of 50 μl of cell suspension in which 5×10⁴ R4/Ba/F3 cells were suspended in RPMI1640 medium containing 10% FBS and 1% Antibiotic G-418 Sulfate. The resultant mixture was stirred lightly. Finally, 10 μl of heparin/10% FBS/1% Antibiotic G-418 Sulfate/RPMI1640 medium (final heparin concentration: 5 μg/ml) was added. Then, the cell was cultured in a carbon dioxide incubator at 37° C. under 5% CO₂ for 72 hr. To determine the growth of R4/Ba/F3 cell, 10 μl of Cell Counting Kit-8 (manufactured by Dojindo Laboratories and sold by Wako Pure Chemical Industries)/PBS solution was added to each well after 72-hr culture, followed by culturing for another 3 hrs, and the coloring at 450 nm which was proportionate to the yield of WST-8 formazan was measured.

In the measurement, 10 μl of FGF18 (PeproTech; 100-28) solution (final FGF18 concentration: 3 ng/ml) was used as a positive control. As a negative control, 10 μl of water or ethanol solution (final concentration of ethanol was adjusted to 1% or less) used for preparing test solutions was used. Cell proliferation rate (%) was calculated by measuring the absorbance obtained when the test solution was added. The absorbance obtained when FGF18 (final concentration: 3 ng/ml) as the positive control had been added was taken as 100%; and the absorbance obtained when water or ethanol solution as the negative control had been added was taken as 0%.

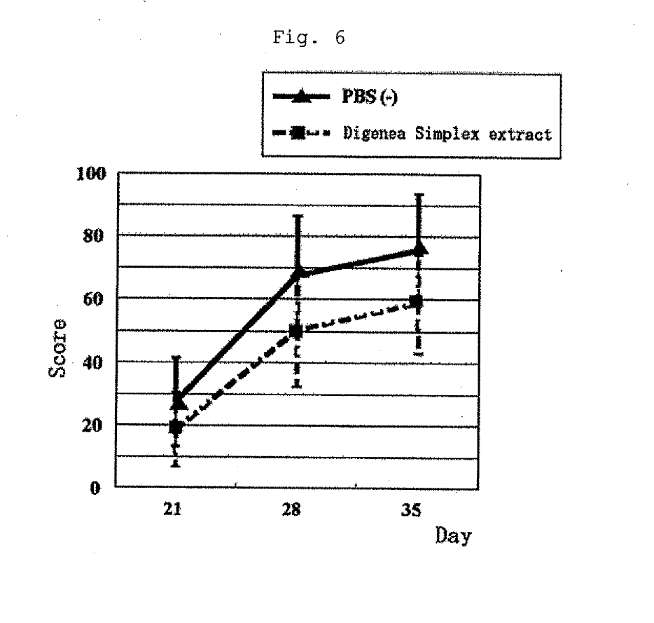

The results on Digenea simplex extracts are shown in FIG. 5. When Digenea simplex extract of final concentration 8.3% was used as a test solution, the amount of coloring increased. Thus, it was confirmed that this Digenea simplex extract has an FGF18-like cell growth promoting activity.

In FIG. 5, Digenea simplex extract exhibited a stronger cell growth promoting effect at concentrations 0.083% and 0.83%, compared to the case where FGFR4/BaF3 strain was cultured in the presence of FGF18 alone. Thus, Digenea simplex extract of low concentrations has an FGF18 activity promoting effect.

In short, Digenea simplex extract is both an FGF-like active substance and a substance that promotes FGF18 activity.

### Example 6

In order to examine the in vivo effect of the FGF18-like active substance, a test was performed on C3H/HeN mice in telogen phase of the hair cycle.

In vivo Analysis of the FGF18-like Active Substance of the Invention

In the same manner as described in Example 5, 30 ml of distilled water was added to 2.0 g of dried Digenea simplex (Ryukyu Marine Product Processing Plant, or Naga Nori Shokai). The resultant mixture was boiled for 20 min. After the extract returned to room temperature, it was centrifuged at 12,000 rpm for 10 min to give a supernatant. This supernatant was filtered through a 0.45 mm Millex HV sterilization filter to make a test solution. The thus obtained Digenea simplex extract was administered subcutaneously to the dorsal skin of C3H/HeN mice in telogen phase of the hair cycle. After anesthetizing, dorsal hair of 7 week-old C3H/HeN male mice was depilated gently with a hair clipper. After depilation, 100 of the Digenea simplex extract as test solution or PBS(−) (physiological phosphate buffer) was injected subcutaneously into 5 mice per group (day 0). In a similar manner; subcutaneous injection was performed again at day 4. The state of hair regrowth in the depilated area in the back of mice was observed with eyes at day 21, day 28 and day 35, to thereby give hair regrowth scores. Each state was scored as follows: 1) pigmentation: 1 point; 2) short hair 2 points; 3) usual hair: 3 points. The ratio of the area of each hair rebirth state to the total depilated area was determined in %. Then, hair regrowth score was calculated by the formula described below. According to this calculation method, the score is 100 when the total depilated area has been recovered to usual hair state.

Hair regrowth score=[ratio of pigmentation area (%)×1+ratio of short hair area (%)×2+ratio of usual hair area (%)×3]/3

The state of hair regrowth in the depilated area in the back of mice was observed at day 21 (3w), day 28 (4w) and day 35 (5w) after the first subcutaneous injection. As a result, as shown in FIG. 6, Digenea simplex extract administered group exhibited lower hair regrowth scores than PBS(−) administered group. Thus, it was demonstrated in vivo that Digenea simplex extract is capable of inhibiting hair growth.

As described so far, the effect of Digenea simplex as a hair regrowth inhibiting agent has been demonstrated.

### Example 7

A formulation of a hair shampoo of the present invention comprising a Digenea simplex extract and a method of preparing the shampoo are described below. A hair shampoo was prepared according to the following formulation and preparation method.

### (Formulation)

| Component | Weight % |
|---|---|
| 1. Diluted solution obtained in Example 5 | 0.1 |
| 2. Sodium laurylether sulfate ethanol | 20 |
| 3. Sodium lauryl sulfate | 10 |
| 4. 1,3-Butylene glycol | 1 |
| 5. Flavor | proper quantity |
| 6. Purified water | to make the total 100 |

### (Preparation Method)

The components listed above were heated to 80° C., mixed by stirring and then cooled under stirring. Thus, the shampoo of the present invention was prepared.

### Example 8

A formulation of a hair liquid of the present invention comprising a Digenea simplex extract and a method of preparing the hair liquid are described below.

A hair liquid was prepared according to the following formulation and preparation method.

### (Formulation)

| Component | Weight % |
|---|---|
| 1. Diluted solution obtained in Example 5 | 0.1 |
| 2. Ethanol | 40 |
| 3. Glycerol | 1 |
| 4. Flavor | proper quantity |
| 5. Purified water | to make the total 100 |

### (Preparation Method)

The components listed above other than purified water were dissolved by stirring and then purified water was added. Thus, the hair liquid of the present invention was prepared.

### Example 9

A formulation of a hair cream of the present invention comprising a Digenea simplex extract and a method of preparing the hair cream are described below.

A hair cream was prepared according to the following formulation and preparation method.

### (Formulation)

| Component | Weight % |
|---|---|
| 1. Diluted solution obtained in Example 5 | 0.1 |
| 2. Liquid paraffin | 40 |
| 3. Vaseline | 1 |
| 4. Cetostearyl alcohol | 1 |
| 5. Methyl polysiloxane | 1 |
| 6. Methyl paraoxybenzoate | 0.2 |
| 7. Propylene glycol | 5 |
| 8. Flavor | proper quantity |
| 9. Purified water | to make the total 100 |

### (Preparation Method)

The components listed above were mixed by stirring to thereby prepare the hair cream of the present invention.

### INDUSTRIAL APPLICABILITY

According to the present invention, effective hair growth inhibiting agents and hair removing agents are provided. By incorporating these agents, it is possible to provide shampoos and hair liquids with hair growth inhibiting activity, as well as depilating creams for removing unwanted hair from the skin.

1-6. (canceled)

7. A method of screening according to claim 17, said screening comprising the following steps (a) to (c):
(a) compulsively expressing at least one FGF receptor gene selected from FGFR1c, FGFR2c, FGFR3c and FGFR4 on the surface of a cell by means of genetic engineering and culturing the cell;
(b) bringing a test substance into contact with the cell system obtained in step (a) having the FGF receptor on cell surfaces; and
(c) selecting those test substances which exhibited FGF18 like cell growth promoting activity in step (b).

8. A method of screening according to claim 17, said method screening comprising the following steps (a) to (c):
(a) compulsively expressing four FGF receptor genes, FGFR1c, FGFR2c, FGFR3c and FGFR4, on respective cell surfaces by means of genetic engineering and culturing the four cells;
(b) bringing a test substance into contact with the four cell systems obtained in step (a) having the FGF receptors on cell surfaces; and
(c) selecting those test substances which, similar to EGF18, exhibit markedly higher cell growth promoting activity on the FGFR3c-expressing cell and the FGFR4-expressing cell than on the FGFR1c-expressing cell and the FGFR2c-expressing cell at the same concentration.

9. The method according to claim 7, wherein the cell on whose surface the FGF receptor is compulsively expressed is mouse IL-3-dependent Ba/F3 cell strain.

10. A method of screening according to claim 17, said screening comprising the following steps (a) to (c):
(a) compulsively expressing at least one FGF receptor gene selected from FGFR1c, FGFR2c, FGFR3c and FGFR4 on the surface of a cell by means of genetic engineering and culturing the cell;
(b) bringing, together with FGF18, a test substance into contact with the cell system obtained in step (a) having the FGF receptor on cell surfaces; and
(c) selecting those cell systems which exhibited a higher cell growth promoting activity in step (b) than when FGF18 was allowed to act singly.

11. The method according to claim 10, wherein the FGF receptor is FGFR3c.

12. The method according to claim 10, wherein the FGF receptor is FGFR4.

13. The method according claim 10, wherein the cell on whose surface the FGF receptor is compulsively expressed is mouse IL-3-dependent Ba/F3 cell strain.

14. A method of screening according to claim 17, said screening comprising the following steps (a) to (d):
(a) preparing an experimental animal or a cultured animal cell capable of expressing FGF18 to an observable extent;
(b) bringing a test substance into contact with or administering the same to the experimental animal, or bringing the same into contact with the cultured animal cell;
(c) monitoring the expression of FGF18 in the experimental animal or the cultured animal cell after step (b); and
(d) selecting those test substances which have a function of promoting the expression of FGF18.

15. The method according to claim 14, wherein the expression of FGF18 is monitored in step (c) by extracting mRNA from the experimental animal or the cultured animal cell after step (b) and then analyzing the mRNA level of expressed FGF18; and those test substances which have a function of promoting the expression of FGF18 are selected in step (d) by selecting systems that exhibited higher levels of FGF18 mRNA than when FGF18 was not allowed to act.

16. The method according to claim 8, wherein the cell on whose surface the FGF receptor is compulsively expressed is mouse IL-3-dependent Ba/F3 cell strain.

17. A method of screening from test substances, comprising:
screening for a substance that promotes the activity or expression of FGF18 or an FGF18-like active substance as a candidate for a hair growth-inhibiting agent or a hair removing agent.
